(19) United States
(12) Patent Application Publication
(10) Pub. No.: US 2014/0276830 A1
(43) Pub. Date: Sep. 18, 2014

(54) BONE STAPLES AND METHODS OF USE THEREFOR AND MANUFACTURING THEREOF

(71) Applicant: Daniel F. Cheney, San Antonio, TX (US)

(72) Inventor: Daniel F. Cheney, San Antonio, TX (US)

(21) Appl. No.: 13/804,756

(22) Filed: Mar. 14, 2013

(51) Int. Cl. A61B 17/064

(52) U.S. Cl. CPC A61B 17/0642; A61B 17/0644
USPC 606/75; 264/138

(57) ABSTRACT

A staple includes a staple bridge and a plurality of staple legs adjoined to the staple bridge. The staple bridge is a shape memory metal, and includes a bridge-shape movable between a first shape and a second shape with no substantial plastic deformation of the staple bridge. The plurality of staple legs are shape memory metal and movable between a first shape and a second shape. The staple stores mechanical energy when the bridge and the plurality of legs move from their first shape to their second shape. The staple releases the stored mechanical energy without a change in temperature of the staple when the bridge and the plurality of legs moved from their second shape to their first shape.

### BACKGROUND OF THE INVENTION

1. Field of the Invention

The present invention relates to staples used for the fixation of bone and soft tissue of the musculoskeletal system and methods of use and manufacturing therefore. More particularly, but not by way of limitation, the present invention relates to staples that change shape through their metallurgic properties and their interaction with mechanical instruments to pull together and compress bone.

2. Description of the Related Art

Bone staples consist of staples bent with instrumentation (bendable staples), shape memory alloy staples sensitive to heat energy (memory staples), and mechanical elastic bone staples (elastic staples). Bendable staples are subject to plastic (permanent) deformation during use. Bones for fixating are aligned, and the bendable staple is inserted between the bones. The bendable staple is then plastically deformed using instrumentation such as pliers or forceps such that the bendable staple maintains the bones fixated together. While bendable staples operate adequately, they do not store mechanical energy and thus cannot continuously apply force to the fixated bones during the healing process.

Memory staples include a first final shape and the ability to be mechanically deformed to a second shape. Memory staples further are subject to elastic (recoverable) deformation during use in that, upon the application of heat energy, memory staples elastically deform from their second shape to their first final shape. Bones for fixating are aligned, and the memory staple is inserted between the bones. The memory staple is then elastically deformed to its first final shape due to the application of heat energy such that the memory staple maintains the bones fixated together. Memory staples store mechanical energy and thus pull together and compress the bones during the healing process. However, due to their transition as a result of heat energy, medical procedures employing memory staples are more time consuming, difficult to perform, and have increased costs.

Elastic staples include a first final shape and the ability to be mechanically deformed by instrumentation to a second shape. Elastic staples further are subject to elastic (recoverable) deformation during use in that, upon release from the instrumentation, elastic staples elastically deform from their second shape to their first final shape. Bones for fixating are aligned, and the memory staple is inserted between the bones using instrumentation that also mechanically deforms the elastic staple to its second shape. After insertion the elastic staple is released from the instrumentation, whereupon the elastic staple elastically deforms to its first final shape such that the elastic staple maintains the bones fixated together. Elastic staples store mechanical energy and thus continuously apply force to the fixated bones during the healing process. However, elastic staples typically include a bridge that transitions from a second shape to a first final shape or legs that transitions from a second shape to a first final shape. Elastic staples therefore are not optimal in pulling together and compressing bones during the healing process.

Accordingly, a staple that is easy and cost effective to manufacture and that elastically transitions independent of temperature at both its bridge and legs would be an improvement in staples used for bone and soft tissue fixation.

### SUMMARY OF THE INVENTION

In accordance with the present invention, a staple is formed in a first shape and movable to second shape upon the application of mechanical energy to the staple. The movement of the staple from its first shape to its second shape stores mechanical energy in the staple, and at least some of the mechanical energy stored in the staple is due to elastic deformation of the staple. The staple is constrained and maintained in the second shape such that the stored mechanical energy moves the staple substantially toward the first shape when the staple is unconstrained. The staple includes a bridge and at least a first leg and a second leg adjoined to the bridge. The bridge and first and second legs are a shape memory metal, and the bridge and first and second legs are movable between a first shape and a second shape with no substantial plastic deformation of the bridge and the first and second legs. The staple stores mechanical energy when the bridge and the first and second legs move from their first shape to their second shape. The staple releases the stored mechanical energy without a change in temperature of the staple when the bridge and the first and second legs move from their second shape to their first shape. The staple may comprise nitinol such that the first shape is in the austenite form and the second shape comprises shape is in the stress induced martensite form.

The staple when is in the second shape stores the mechanical energy predominately where the first and second legs adjoin the staple bridge and in the curvature of the bridge. The staple is operable for pulling together and compressing bone when the staple moves from the second shape to the first shape. Conversely, the staple is operable for pulling apart and placing bone under tension when the staple moves from the second shape to the first shape.

A first bone structure is connected with a second bone structure by inserting the first leg in the first bone structure and the second leg in the second bone structure, and releasing the bridge and the first and second legs such that the bridge and first and second legs move from their second shapes to their first shapes without a change in temperature in the staple. Connecting a first bone structure with a second bone structure further includes drilling a first hole in the first bone structure before inserting the first leg into the first bone structure and drilling a second hole in the second bone structure before inserting the second leg into the second bone structure. Nevertheless, the first leg may be inserted into an undrilled portion of the first bone structure, and the second leg may be inserted into an undrilled portion of the second bone structure.

In connecting a first bone structure with a second bone structure, the bridge is operable to deform when moved between the first shape and the second shape. In particular, the deformation of the bridge may include non-plastic deformation, plastic deformation, elastic deformation, and pseudo elastic deformation of the bridge when moved between the first shape and the second shape. The staple may be non-parallel when the bridge is in the first shape and parallel when the bridge is in the second shape. As such, the staple is operable to deform when moved between the non-parallel shape and the parallel shape such that the deformation of the staple is a non-plastic deformation, plastic deformation, elastic deformation, and pseudo elastic deformation of the staple when moved between the non-parallel shape and the parallel shape.

A method of manufacturing a staple movable between a first shape and a second shape includes cutting a first view of the staple from a first face of a rod, cutting a second view of the staple from a second face of the rod, and cutting a third view of the staple from a third face of the rod, thereby forming the staple in a first shape. The method further includes moving the staple into the second shape with no substantial plastic deformation of the staple, and constraining the staple in the second shape.

It is an object of the present invention to provide a staple movable from a first shape to a second shape with no substantial plastic deformation of the staple such that the staple the stores mechanical energy.

It is a further object of the present invention to provide a staple movable from a second shape that stores mechanical energy to a first shape independent of a change in temperature in the staple.

It is still further an object of the present invention to cut a staple in a first shape using a three-dimensional cutting technique.

Still other objects, features, and advantages of the present invention will become evident to those of ordinary skill in the art in light of the following. Also, it should be understood that the scope of this invention is intended to be broad, and any combination of any subset of the features, elements, or steps described herein is part of the intended scope of the invention.

### BRIEF DESCRIPTION OF THE DRAWINGS

FIG. 1A is a perspective view illustrating a first embodiment of a staple in a first closed position.

FIG. 1B is a top view illustrating a first embodiment of a staple in a first closed position.

FIG. 1C is a front view illustrating a first embodiment of a staple in a first closed position.

FIG. 2A is a perspective view illustrating a first embodiment of a staple in a second open position.

FIG. 2B is a top view illustrating a first embodiment of a staple in a second open position.

FIG. 2C is a front view illustrating a first embodiment of a staple in a second open position.

FIG. 3A is a perspective view illustrating a second embodiment of a staple in a first closed position.

FIG. 3B is a perspective view illustrating a second embodiment of a staple in a second open position.

FIG. 4 is a perspective view illustrating a bar or rod utilized in a three dimensional manufacturing method for a staple.

### DETAILED DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. It is further to be understood that the figures are not necessarily to scale, and some features may be exaggerated to show details of particular components or steps.

As discussed and described herein, embodiments of the present inventions include staples and method of use including staples in which the staples are able to move between two shapes, with, generally, one shape being a “parallel” shape and the other shape being a “non-parallel” shape. A staple has a “parallel” shape when the legs of the staple are in a substantially parallel orientation, as opposed to a convergent orientation or a divergent orientation. A staple has a “non-parallel” shape when the legs of the staple are in a convergent orientation or a divergent orientation. The staples move between a parallel and non-parallel shape in order to secure bones or bone fragments together.

FIGS. 1A-2C describe an S-shaped embodiment of a staple 10. The staple 10 includes a bridge 20 and legs 30 formed integrally at corners 40 and 41. The legs 30 further include tips 31 and 32 and bone retention notches 33 and 34. The tips 31 and 32 of the legs 30 may form a shape that is rounded for insertion into drill holes or the tips 31 and 32 are pointed for impaction into bone. The retention notches 33 and 34 are designed to grip bone and prevent slippage once the staple 10 has been inserted into bone. By way of example the staple 10 has two legs 40, however, those of ordinary skill in the art will recognize that the staple 10 may include more than two legs 30.

The staple 10 is designed to move between a second parallel shape (i.e., the legs 30 of the staple 10 are substantially parallel) and a first convergent shape (i.e., the legs 30 of the staple 10 are in a convergent orientation). When the staple 10 is designed to move between a second parallel shape and a first convergent shape the staple 10 is referred to as a “convergent staple.” The non-parallel configuration of the staple 10 wherein the legs 30 of the staple 10 converge is the referred to as the “closed” shape of the staple 10. Likewise, the parallel configuration of a staple 10 wherein the legs 30 of the staple 10 are substantially parallel is referred to as the “open” shape of the staple 10.

In addition, the staple 10 may also be designed to move between a second parallel shape (i.e., the legs 30 of the staple 10 are substantially parallel) and a first divergent shape (i.e., the legs 30 of the staple 10 are in a divergent orientation). When the staple 10 is designed to move between a second parallel shape and a first divergent shape the staple 10 is referred to as a “divergent staple.” The non-parallel configuration of a staple 10 wherein the legs of the staple 10 diverge is referred to as the “open” shape of the staple 10. Likewise, the parallel configuration of a staple 10 wherein the legs 30 of the staple 10 are substantially parallel is referred to as the “closed” shape of the staple 10.

A staple 10 is considered in an open shape or a closed shape dependent upon the orientation of legs 30 and whether the staple 10 is a “convergent staple” or a “divergent staple.” The “open” shape of a convergent staple and the “closed” shape of a divergent staple are the circumstances in which the legs of the staple have a substantially parallel orientation. A convergent staple thus moves from its open shape to its closed shape when the legs of the convergent staple move from the substantially parallel orientation to a convergent orientation. The divergent staple thus moves from its closed shape to its open shape when the legs of the divergent staple move from a substantially parallel orientation to a divergent orientation.

The staple 10 is designed to internally store mechanical energy in its metallic structure and expend this recoverable energy to change the shape of the staple 10 or apply force to bone. In metals that exhibit linear elastic deformation, the energy is stored as molecular bonds in the metallic structure are strained but not broken. Elastic deformation strains and rearranges molecular bonds to store mechanical energy. This energy is recovered when the metal grossly changes shape as a result of its crystalline structure transitions from martensite to austenite. Generally, the mechanical energy is stored when the staple 10 is in a parallel shape (i.e. an open shaped convergent staple or a closed shaped divergent staple), and the mechanical energy is recovered when the staple 10 moves toward its non-parallel shape (i.e., a closed shape convergent staple or an open shaped divergent staple).

FIGS. 1A-1C illustrate the staple 10 according to a preferred embodiment wherein the staple 10 is a convergent staple in a first closed shape. In the first closed shape the bridge 20 of the staple 10 is undulated and contracted, and the legs 30 are angled together with the tips 31 and 32 of the legs 30 converging. FIGS. 2A-2C illustrate the staple 10 in a second open shape. In the second open shape of the staple 10 the undulation of the bridge 20 has been lengthened and the legs 30 and the bridge 20 have been strained, predominately at the corners 40 and 41 adjoining the bridge 20 so that the legs 30 are parallel with one another. This strain in the legs 30, the bridge 20 and the corners 40 and 41 stores energy by 1) stretching molecular bonds within their recoverable elastic range and/or by 2) creating recoverable stress induced martensite in its structure if fabricated from a shape memory metal, such as nitinol.

With respect to the former, this linear elastic behavior (caused by the stretching of molecular bonds) is common to spring tempered metals, including, but not limited to, stainless steel, titanium, nickel-chromium alloys (such as Inconel alloys), memory shaped material (such as nitinol), and other alloys. This behavior is referred to as “elastic deformation” in that once the strain is removed, the molecules will no longer remained stretched and substantially return to their original position (thus releasing the stored energy).

With respect to the latter, this change of structure occurs in certain materials, such as shape memory metals (like nitinol) that can transform from one structure form to another structure form. Shape memory materials, like nitinol, have an austenite phase (cubic B2 structure) and a martensite phase (monoclinic B19 structure). Strain in the bridge 20, the legs 30 and corners 40 and 41 can cause stress induced transformation of the shape memory metal such that a portion of the shape metal material (such as in the bridge 20, legs 30, and the corners 40 and 41) will transform from austenite to martensite. This behavior is referred to as “pseudo elastic deformation” in that once the strain is removed, the shape memory material will return to austenite, and the material will substantially return to its original position (thus releasing the stored energy). When pseudo elastic deformation (and elastic deformation) occurs before any substantial conventional plasticity, the shape memory material is referred to as exhibiting “super elasticity.”

Over-stretching can lead to formation of permanent deformation that renders the material incapable of returning completely to its original shape (or for reverting to austenite). This behavior is referred to as “plastic deformation” and also “permanent deformation” in that when the strain is removed the material that is permanently deformed will not substantially return to its original shape. The combined behavior of elastic deformation and pseudo elastic deformation are sometimes referred to collectively as “non-plastic deformation” and “non-permanent deformation.”

It should be noted that a material can be plastically deformed in some portions and non-plastically deformed in other portions. Indeed, the non-plastic deformations may itself be a combination of elastic deformations and pseudo-elastic deformations. Thus, a material under strain could deform having a plastic deformation component, a non-plastic deformation component, and a pseudo elastic deformation component. For materials that do not change phase under stress, the pseudo elastic deformation component would basically be zero.

As the amount of non-particle deformation component increases the amount of plastic deformation component increases versus the amount of plastic deformation component, the more the material will tend to move toward its original shape (i.e., return toward its original shape) when the strain is removed.

For instance, when the plastic deformation component is insubstantial (i.e., the material will substantially return to its original shape when the strain is removed), the deformation components are substantially all non-plastic deformation components. In the present application, there is “no substantial plastic deformation” when the material is substantially able to return to its original configuration after the strain is removed (i.e., the plastic deformation component is basically insubstantial when compared to the non-plastic deformation component). In some embodiments of the present invention, the strain in the legs 30, the bridge 20, and the corners 40 and 41 store energy with no substantial deformation of the staple 10 (including no substantial deformation of the legs 30, bridge 20, and the corners 40 and 41).

Alternatively, for instance, the deformation may include both a substantial plastic deformation component. A material could be plastically deformed to a degree that it cannot return to its original shape once the strain is removed; but, the material could still tend to move back toward (but not completely) to its original shape when the strain is removed. Strain in the legs 30, the bridge 20, and the corners 40 and 41 could store energy due to non-plastic deformation, and substantial elastic/or pseudo elastic deformation can occur even when there is substantial plastic deformation of the staple 10. Thus, in some embodiments of the present invention, the strain in the legs 30, the bridge 20, and the corners 40 and 41 store energy even when there is substantial deformation of the staple 10 (including substantial deformation of the legs 30, the bridge 20 and/or the corners 40 and 41). Generally, such materials are not shaped memory, but usually other materials that exhibit substantial elastic deformation components even when deformed in conjunction with plastic deformation of the material.

FIGS. 3A and 3B describe a staple 50 which is convergent and an alternate embodiment of the staple 10. The staple 50 includes a bridge 60 that is O-shaped and legs 70 formed integrally at corners 80 and 81. The legs 70 further include tips 71 and 72 and bone retention notches 73 and 74. The tips 71 and 72 of the legs 70 may form a shape that is rounded for insertion into drill holes or the tips 71 and 72 that are pointed for impaction into bone. The retention notches 73 and 74 are designed to grip bone and prevent slippage once the staple 50 has been inserted into bone. By way of example the staple 50 has two legs 70, however those of ordinary skill in the art will recognize that the staple 50 may include more than two legs 70.

FIG. 3A illustrates a first closed shape wherein the bridge 60 is contracted and the legs 70 are angled together with the tips 71 and 72 of the legs 70 converging. FIG. 3B illustrates a second open shape of the staple 50 wherein the bridge 60 has been lengthened and the legs 70 are parallel. The second open shape is the implanted shape of the staple 50 such that, when the staple 50 is placed into a bone and released, the stored mechanical energy within the metallic structure of the staple 50 causes the legs 70 to move toward one another and the bridge to contract and pull together and compress the bone.

The staple 10 and the staple 50 can be manufactured using various techniques, and, for the sake of disclosure, manufacture of the convergent staple 10 will be described herein. Nevertheless, one of ordinary skill in the art will recognize that divergent staples as well as the staple 50 may be manufactured using the techniques described herein.

The staple 10 may be manufactured from shape changing nitinol cut from wire, bent over appropriate fixtures, and heat treated to form the first closed shape for the staple 10. Unfortunately this technique may result in stresses, crimps, and localized deformations at the bend locations that negatively impact the performance of the staple 10.

The staple 10 also may be manufactured from plate. Using EDM, water jet, or other cutting technology, the flattened shape of the staple 10 is cut, and features added through bending over appropriate fixtures as with wire. Similar to wire, the bending steps may result in stresses, crimps, and localized deformations at the bend locations of the staple 10.

In a preferred method illustrated in FIG. 4, the staple 10 may be manufactured in a three dimensional cutting technique from a bar or rod 80 of solid material using EDM, water jet, laser machining, or other cutting technology. When manufactured three dimensionally from a bar or rod 80 of solid material, the staple 10 may be cut into its first closed shape or the staple 10 may be cut into an intermediate shape between the first closed shape and the second open shape. The bar or rod 80 is held in an appropriate fixture of a cutting machine. The staple 10 is first cut in a top view from a top face 81 of the bar or rod 80. The bar or rod 80 is then rotated 90 degrees, and the staple 10 is cut in a front view from a front face 82 of the bar or rod 80. The bar or rod 80 is again rotated 90 degrees, and the staple 10 is cut in a side view from a side face 83 of the bar or rod 80. Upon completion of the third and final cut, the staple 10 is ready for removal from the bar or rod 80, which typically is the staple 10 dropping from the bar or rod 80 due to gravity. Using this method, the staple 10 is produced in quantity and in its first closed shape with no deformation or stress due to bending. The three dimensional manufacturing method of the preferred embodiment for the shape changing staples 10 and 50 accordingly significantly simplifies manufacturing, reduces cost, and minimizes staple performance variation.

After the cutting of the staple 10 using the three dimensional manufacturing method of the preferred embodiment, the staple 10 must be moved to its second open shape prior to use in a surgical procedure. In particular, as illustrated in FIG. 2C, the legs 30 are strained to become the parallel, while, simultaneously, the S-shaped bridge 20 is strained to become elongated. This straining stores mechanical energy in the metal matrix of the staple 10, and results in a situation where the staple 10 would spontaneously return to its first closed shape if released. The staple 10 accordingly is used in combination with instrumentation that maintains the staple 10 strained in its second open shape. While the instrumentation may be employed at the location of a surgical procedure just prior to use of the staple 10, the instrumentation is typically used during manufacture of the staple 10 such that the staple remains constrained in its second open shape during shipping, handling and implantation of the staple 10.

Examples of instrumentation suitable to constrain the staple 10 in its second open shape during shipping, handling and implantation include but are not limited to the instrumentation disclosed in U.S. Pat. No. D675,734 S or US Design patent application Ser. No. 29/442,289. In particular, the bridge 20 of the staple 10 is strained to become the elongated and the legs 30 are strained to become the parallel followed by the insertion of the staple 10 into the instrumentation such that the staple 10 remains in its second open shape. Moreover, it should be understood that staple 10 may be incorporated into an orthopedic fixation system such as that disclosed in U.S. patent application Ser. No. 13/385,387, wherein the staple 10 is delivered sterilized in sterile packaging.

The staples 10 and 50 are uniquely suited for fixation of materials that have a tendency to benefit from compression or shrink and withdraw so that the stapled structures lose contact. Without limiting the scope of the invention the staples 10 and 50 are used for bone fixation. In bone surgery, fragments, separated segments, and segments requiring fixation are pulled together by the staples 10 and 50 because they are inserted so that at least one of a plurality of legs is placed in two or more bone segments. This method of surgical use is common to bone staples.

The staples 10 and 50 exert bone compression force that is not temperature dependent. This provides tremendous advantage for the surgeon and patient over prior art nitinol shape changing implants. Temperature independence solves problems with the prior art nitinol staples because the staples 10 and 50 apply consistent force prior, during, and following implantation. Body temperature staple force changes as the operative wound warms from near room temperature to body temperature. This force increase occurs after the wound is closed and without the knowledge of the surgeon can create fracture or deformity.

The staples 10 and 50 are held by the instrumentation to store the mechanical energy within the structure of the staples 10 and 50. This mechanical energy is stored through the elasticity of the metal if stainless steel or other linear elastic metal, or in a stress induced martensitic state if fabricated from nitinol or other material that exhibits this behavior. Once removed from the instrumentation the staples 10 and 50 spontaneously act to return to their first closed shape. This shape change pulls together and compresses bone.

Continuing the example specific to the staple 10, a surgeon during use in a surgical procedure inserts the legs 30 of the staple 10 into a bone across a fracture or joint requiring fixation. The tips 31 and 32 of the legs 30 are either forced into bone through impaction or inserted into drilled holes matched to the diameter and separation between the legs 30 of the staple 10. Once the legs 30 of the staple 10 are partially in bone the staple 10 is removed from the instrumentation. As the staple 10 is removed from the instrumentation, the legs 30 of the staple 10 begin to move inward pulling bone together and exerting compression forces. As the staple 10 continues to advance into bone, the elastic energy acting to transition the staple 10 from its second open shape to its first closed shape is transferred from the instrumentation and to bone. This elastic energy converts to work and pulls the bone together applying a residual compression force. Once the staple 10 is fully removed from the instrumentation, the staple 10 applies its full force to pull together and compress bone. The transfer of shape changing forces from the instrumentation to bone can be controlled by the staple 10 and instrumentation designs or the rate at which the surgeon removes the staple 10 from the instrumentation. While the foregoing methods for the sake of example utilized the staple 10, one of ordinary skill in the art will recognize that the methods work equally well and may be employed with the staple 50.

The operation of the staples 10 and 50 allows a novel and cost effective manufacturing technique and results in a stronger and more consistent implant. First, the operation of the staples 10 and 50 are independent of temperature in the range of temperatures expected in clinical use. Thus, tight control of material's crystalline structure transitions temperature is not required. Furthermore, the temperatures are set so that the material is always in its strong and high temperature austenic form. Thus, as long as the austenitic finish temperature is above 20 degrees C, then it will be stable in the operating theater and patient's body. So fine chemistry control and post heat treatments to shift transition temperatures is not required.

To complement the temperature independent operational mode, the implant is cut using three-dimensional inline cutting manufacturing methods from a block of material and not bent from an extruded wire or plate. Since the implant is not bent into a final form, stress concentrations in the material or changes in transition temperature do not occur. Thus, the staples 10 and 50 are stronger and less likely to fail from fatigue loading.

Together the manufacturing steps and the requirement to retain the staples 10 and 50 in the second open shape complement the ability to remove the staples 10 and 50 from the instrumentation and together are designed to support the one operative task the surgeon must perform. That task is the advancement of the legs 30 into bone. The surgeon does not need to compress the staples 10 and 50 with pliers, open the staples 10 and 50 to fit into its drill holes, keep the staples 10 and 50 on ice or heat it with electrical current as is required by prior art. The surgeon needs only to put the tips of the legs of the staples 10 and 50 into bone and advance the staples 10 and 50 using the instrumentation until the staples 10 and 50 are fully implanted. The instrumentation can be pushed by hand or impacted with a mallet to fully seat the staples 10 and 50 within the bone. The instrumentation can be reusable and receive the staples 10 and 50 or disposable and be integral component of the staples 10 and 50.

The staples 10 and 50 illustrated in this application are a significant advancement over the prior art staples in: 1) the method of operation of the staple and its high strength, 2) the method of insertion of the staple, 3) its compressive force temperature independence, 4) its efficient staple retention and delivery system, 5) its compatibility with reusable or single use product configuration 6) its efficient and cost effective manufacturing methods, and 7) its minimization of the steps required to place the device. These advantages are important to musculoskeletal surgery as well as industrial applications for staples.

Although the present invention has been described in terms of the foregoing embodiments, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing detailed description; rather, it is defined only by the claims that follow.

1. A staple, comprising:
(a) a staple bridge, wherein:
(i) the staple bridge comprises a shape memory metal,
(ii) the staple bridge has a bridge-shape such that the bridge can move between a first shape and a second shape with no substantial plastic deformation of the staple bridge, and
(iii) the bridge shape is selected from the group consisting of an S-shaped staple bridge shape and O-shaped staple bridge shape; and
(b) a plurality of staple legs adjoined to the staple bridge, wherein:
(i) the plurality of staple legs comprise the shape memory metal,
(ii) the staple is operable for moving between a non-parallel shape and a parallel shape without substantial plastic deformation of the staple,
(iii) the staple is in the non-parallel shape when the staple bridge is in the first shape and the staple legs are not substantially parallel,
(iv) the staple is in the parallel shape when the staple is in the second shape and the staple legs are substantially parallel,
(v) the staple is operable for storing mechanical energy when the staple is in the parallel shape, and
(vi) the staple is operable for moving substantially to the non-parallel shape when stored mechanical energy is released without a change in temperature of the staple.

2. That staple of claim 1, wherein the staple is operable for use in a medical procedure.

3. The staple of claim 1, wherein the staple is a bone staple.

4. The staple of claim 1, wherein the first shape is a contracted shape of the staple bridge, and the second shape is an elongated shape of the staple bridge.

5. The staple of claim 1, wherein the staple bridge has an S-shaped staple bridge shape.

6. The staple of claim 1, wherein the staple bridge has an O-shaped staple bridge shape

7. The staple of claim 1, wherein:
(a) the non-parallel shape is a convergent shape; and
(b) the staple legs are convergent.

8. The staple of claim 1, wherein
(a) the non-parallel shape is a divergent shape; and
(b) the staple legs are divergent.

9. The staple of claim 1, wherein when the staple is in the parallel shape, the stored mechanical energy of the staple is predominately stored where the plurality of staple legs are adjoined to the staple bridge and in the curvature of the staple bridge.

10. The staple of claim 1, wherein the staple is operable for pulling together and compressing bone when the staple moves from the parallel shape to the non-parallel shape.

11. The staple of claim 1, wherein the staple is operable for pulling apart and placing bone under tension when the staple moves from the parallel shape to the non-parallel shape.

12. The staple of claim 1, wherein the shape memory metal comprises nitinol.

13. The staple of claim 12, wherein the shape memory metal of the staple in the non-parallel shape is in the austenite form.

14. The staple of claim 12, wherein the shape memory metal of the staple in the parallel shape comprises shape memory metal in the form of stress induced martensite.

15. The staple of claim 1, wherein the shape memory material has a material strength such that the staple is operable for implanting in bone without pre-drilling holes in bone.

16. The staple of claim 1, wherein each of the staple legs have a rounded leg tip.

17. The staple of claim 1, wherein the staple is a sterilized staple.

18. A method for a staple, comprising:
forming a staple in a first shape;
applying mechanical energy to the staple to move the staple to a second shape, wherein at least some of the mechanical energy is stored in the staple due to elastic deformation of the staple;
constraining the staple in the second shape; and
maintaining the staple in the second shape, wherein the mechanical energy is operable for moving the staple substantially toward the first shape when the staple is unconstrained.

19. The method of claim 18, wherein movement of the staple to the second shape does not substantially plastically deform the staple.

20. The method of claim 19, wherein the mechanical energy is operable for moving the staple substantially to the first shape when the staple is unconstrained.

21. The method of claim 18, wherein the staple is operable for use in a medical procedure.

22. The method of claim 18, wherein the staple is a bone staple.

23. The method of claim 18, wherein the step of applying mechanical energy occurs before the step of constraining the staple.

24. The method of claim 18, wherein the step of applying mechanical energy occurs while performing the step of constraining the staple.

25. The method of claim 18, wherein the step of forming the staple comprises cutting the staple in the first shape.

26. The method of claim 25, wherein the staple is cut from a rod of material.

26. The method of claim 26, wherein the staple is cut using a three-dimensional cutting technique.

27. The method of claim 26, wherein the three-dimensional cutting technique is selected from the group consisting of milling techniques, electro discharge techniques, water jet techniques, laser machining techniques, and combinations thereof.

28. The method of claim 27, wherein the three-dimensional cutting technique, comprises:
cutting a first view of the staple from a first face of the rod;
cutting a second view of the staple from a second face of the rod; and
cutting a third view of the staple from a third face of the rod, thereby forming the staple in the first shape.

29. The method of claim 18, wherein the staple is formed from a metal selected from the group consisting of stainless steel, titanium, nitinol, and their alloys, and combinations thereof.

30. The method of claim 18, wherein the staple is formed from a shape memory metal.

31. The method of claim 18, wherein the staple is formed from nitinol.

32. The method of claim 18, wherein the staple is formed from a super elastic metal.

33. The method of claim 18, wherein the first shape is a non-parallel shape and a convergent shape.

34. The method of claim 18, wherein the first shape is a non-parallel shape and a divergent shape.

35. The method of claim 18, wherein the staple comprises a staple bridge adjoined to a plurality of staple legs.

36. The method of claim 35, wherein:
(a) the first shape is a non-parallel shape and a convergent shape;
(b) the staple bridge is contracted and the staple legs are convergent when the staple is in the first shape; and
(c) the staple bridge is elongated and the staple legs are substantially parallel when the staple is in the second shape.

37. The method of claim 35, wherein the step of applying mechanical energy to the staple comprises elongating the staple bridge.

38. The method of claim 35, wherein the step of applying mechanical energy to the staple comprises moving the staple legs from a convergent orientation to a substantially parallel orientation.

39. The method of claim 35, wherein:
(a) the first shape is a non-parallel shape and a divergent shape;
(b) the staple bridge is elongated and the staple legs are divergent when the staple is in the first shape; and
(c) the staple bridge is contracted and the staple legs are substantially parallel when the staple is in the second shape.

40. The method of claim 35, wherein the step of applying mechanical energy to the staple comprises contracting the staple bridge.

41. The method of claim 35, wherein the step of applying mechanical energy to the staple comprises moving the staple legs from a divergent orientation to a substantially parallel orientation.

42. The method of claim 35, wherein the staple-bridge has an S-shaped staple bridge shape.

43. The method of claim 35, wherein the staple-bridge has an O-shaped staple bridge shape.

44. The method of claim 18, wherein:
(a) the step of forming the staple comprises forming the staple from a shape memory metal in austenite form; and
(b) the step of applying mechanical energy to the staple comprises forming stress induced martensite in the staple.

45. The method of claim 40, wherein:
(a) the staple comprises a staple bridge adjoined to a plurality of staple legs;
(b) each of the plurality of staple legs is adjoined to the staple bridge at corners; and
(c) the step of applying mechanical energy to the staple comprises forming stress induced martensite in the staple at a site selected from the group consisting of the staple bridge and the corners.

46. The method of claim 18, wherein the step of constraining the staple comprises positioning the staple in instrumentation that maintains the staple in its second shape.

47. The method of claim 46, wherein:
(a) the staple comprises an S-shaped staple bridge; and
(b) the instrumentation has a shape to maintain the staple having an elongated S-shaped staple bridge.

48. The method of claim 46, wherein:
(a) the staple comprises an O-shaped staple bridge; and
(b) the instrumentation has a shape to maintain the staple having an elongated O-shaped staple bridge.

49. The method of claim 46, wherein:
(a) the staple comprises an S-shaped staple bridge; and
(b) the instrumentation has a shape to maintain the staple having a contracted S-shaped staple bridge.

50. The method of claim 46, wherein:
(a) the staple comprises an O-shaped staple bridge; and
(b) the instrumentation has a shape to maintain the staple having a contracted O-shaped staple bridge.

51. A method of manufacturing a staple movable between a first shape and a second shape, comprising:
cutting a first view of the staple from a first face of a rod;
cutting a second view of the staple from a second face of the rod; and
cutting a third view of the staple from a third face of the rod, thereby forming the staple in a first shape.

52. The method of claim 51, further comprising:
moving the staple into the second shape with no substantial plastic deformation of the staple; and
constraining the staple in the second shape.

53. A method for connecting a first bone structure with a second bone structure, comprising:
providing a staple comprising a bridge and first and second legs;
moving the bridge from a first shape to a second shape with no substantial plastic deformation of the bridge;
moving the first and second legs from a first shape to a second shape with no substantial plastic deformation of the first and second legs;
constraining the bridge and the first and second legs in their second shapes;
inserting the first leg in the first bone structure and the second leg in the second bone structure; and
releasing the bridge and the first and second legs, wherein the bridge and first and second legs move from their second shapes to their first shapes without a change in temperature in the staple, thereby connecting the first bone structure with the second bone structure.

54. The method of claim 53, wherein the staple is used for musculoskeletal surgical repair of the bone structures.

55. The method of claim 53, further comprising:
(a) drilling a first hole in the first bone structure before inserting the first leg into the first bone structure; and
(b) drilling a second hole in the second bone structure before inserting the second leg into the second bone structure.

56. The method of claim 53, wherein:
(a) the first leg is inserted into an undrilled portion of the first bone structure; and
(b) the second leg is inserted into an undrilled portion of the second bone structure.

57. The method of claim 53, wherein:
(a) the bridge is operable to deform when moved between the first shape and the second shape, wherein:
(i) the deformation of the bridge comprises non-plastic deformation of the bridge when moved between the first shape and the second shape,
(ii) the staple is in a non-parallel shape when the bridge is in the first shape, and
(iii) the staple is in a parallel shape when the bridge is in the second shape; and
(b) the staple is operable to deform when moved between the non-parallel shape and the parallel shape, wherein the deformation of the staple comprises non-plastic deformation of the staple when moved between the non-parallel shape and the parallel shape.

58. The method of claim 57, wherein:
(a) the deformation of the bridge further comprises plastic deformation of the bridge when moved between the first shape and the second shape; and
(b) the deformation of the staple further comprises plastic deformation of the staple when moved between the non-parallel shape and the parallel shape.

59. The method of claim 57, wherein:
(a) the deformation of the bridge comprises non-plastic deformation of the bridge without substantial plastic deformation of the bridge when moved between the first shape and the second shape; and
(b) the deformation of the staple comprises non-plastic deformation of the staple without substantial plastic deformation when moved between the non-parallel shape and the parallel shape.

60. The method of claim 59, wherein:
(a) the deformation of the bridge comprises elastic deformation of the bridge when moved between the first shape and the second shape; and
(b) the deformation of the staple comprises elastic deformation of the bridge when moved between the non-parallel shape and the parallel shape.

61. The method of claim 60, wherein:
(a) the deformation of the bridge comprises pseudo elastic deformation of the bridge when moved between the first shape and the second shape; and
(b) the deformation of the staple comprises pseudo elastic deformation of the bridge when moved between the non-parallel shape and the parallel shape.

62. The method of claim 53, wherein the staple comprises a metal selected from the group consisting of stainless steel, titanium, nitinol, and their alloys and combinations thereof.

63. The method of claim 53, wherein the bone staple comprises a shape memory metal.

64. The method of claim 53, wherein the bone staple comprises nitinol.

65. The method of claim 53, wherein the bone staple comprises a super elastic metal.

66. The method of claim 53, wherein the step of constraining the bridge and the first and second legs in their second shapes comprises positioning the staple in instrumentation that maintains the bridge and the first and second legs in their second shapes.

67. The method of claim 66, wherein
(a) the staple comprises an S-shaped staple bridge; and
(b) the instrumentation has a shape to maintain the staple having an elongated S-shaped staple bridge.

68. The method of claim 66, wherein:
(a) the staple comprises an O-shaped staple bridge; and
(b) the instrumentation has a shape to maintain the staple having an elongated O-shaped staple bridge.

69. The method of claim 53, wherein:
(a) the first shape is a non-parallel shape and a convergent shape; and
(b) the staple legs are convergent.

70. The method of claim 53, wherein:
(a) the first shape is a non-parallel shape and a divergent shape; and
(b) the staple legs are divergent.
